(19) United States
(12) Patent Application Publication
(10) Pub. No.: US 2022/0387439 A1
(43) Pub. Date: Dec. 8, 2022

(54) ANTI-INFLAMMATORY PHARMACEUTICAL COMPOSITION COMPRISING PROBIOTICS AND ANTIBIOTICS, AND METHOD USING SAME

(71) Applicant: Min Bo Shim, Seoul (KR)

(72) Inventor: Min Bo Shim, Seoul (KR)

(21) Appl. No.: 17/789,149

(22) PCT Filed: Dec. 21, 2020
(86) PCT No.: PCT/KR2020/018756

§ 371 Date: Jun. 24, 2022

(30) Foreign Application Priority Data
Dec. 31, 2019 (KR) 10-2019-0179806

(51) Int. Cl. A61K 31/546; A61P 29/00; A61K 35/747; A61K 35/742; A61K 35/744; A61K 35/745; A61K 36/064; A61K 31/65; A61K 31/545; A61K 31/43; A61K 9/00; A61P 27/16; A61P 31/04; A61P 31/10

(52) U.S. Cl. CPC A61K 31/546; A61P 29/00; A61K 35/747; A61K 35/742; A61K 35/744; A61K 35/745; A61K 36/064; A61K 31/65; A61K 31/545; A61K 31/43; A61K 9/0046; A61K 9/0019; A61P 27/16; A61P 31/04; A61P 31/10; A61K 2035/115

(57) ABSTRACT

This application relates to a pharmaceutical composition for anti-inflammation, including a probiotic and an antibiotic, and a method of preventing or treating inflammation by using the same. In one aspect, the method includes locally administrating a probiotic and an antibiotic to an inflamed area of a subject.

### TECHNICAL FIELD

The present disclosure relates to a pharmaceutical composition for anti-inflammation, including a probiotic and an antibiotic and a method of preventing or treating inflammation by using the same.

### BACKGROUND ART

Inflammation occurs biochemically due to various causes such as bacterial or fungal infection, and is an immune response that occurs locally, when cells or tissues are damaged or destroyed by excessive inflammatory responses, to minimize the damage or restore the destroyed area to its original state. In normal cases, this inflammatory response reduces incidence factors or restores the damaged area to a normal structure by regeneration, but an excessive inflammatory response beyond the normal range causes various diseases, including chronic inflammation.

Non-steroidal anti-inflammatory drugs (NSAIDs), which are widely used agents for the treatment of most inflammatory diseases, exhibit anti-inflammatory action by inhibiting the activity of an enzyme involved in the biosynthesis of prostaglandin from arachidonic acid called cyclooxygenase (COX), but causes serious side effects such as gastrointestinal disorders, liver disorders, and renal disorders, in addition to the main therapeutic action, and thus the long-term use thereof is limited.

In addition, antibiotics are used to treat bacterial or fungal infection, but it is difficult to treat antibiotic-resistant bacteria only with antibiotics. In particular, multidrug-resistant bacteria that do not respond to several types of antibiotics at the same time are serious in that the fatality rate is high.

Meanwhile, a method of preventing or treating inflammatory diseases such as acute otitis media by administering probiotics was developed (Korean Patent Publication No. 10-2007-0052246), or novel lactic acid bacteria having inflammatory response inhibitory efficacy were developed (Korean Patent Publication No. 10-2019-0082739), but these were developed as formulations for oral or intraperitoneal administration. Thus, it is difficult to expect rapid therapeutic effects.

Therefore, there is a need to develop a composition for effectively treating inflammation, particularly inflammation caused by bacterial or fungal infection, and a method of preventing or treating inflammation by using the same.

### DESCRIPTION OF EMBODIMENTS

### Technical Problem

One aspect provides a pharmaceutical composition for anti-inflammation. Another aspect provides a method of preventing or treating inflammation.

### Technical Solution to Problem

One aspect provides a pharmaceutical composition for anti-inflammation, including a probiotic and an antibiotic.

The term “probiotics” refers to live bacteria that have a beneficial effect on the health of an individual. Probiotics may be strains that have a beneficial action on the intestinal environment when ingested and reaching the intestines. Probiotics can produce lactic acid, making the surrounding environment acidic. Bacteria or fungi that cannot withstand an acidic environment are killed, and beneficial bacteria that grow well in an acidic environment can further proliferate. The proliferated probiotics can change the flora of a site to which the probiotics are administered.

The probiotic may be a bacterium of a genus selected from the group consisting of the genus Lactobacillus, the genus Lactococcus, the genus Bacillus, the genus Enterococcus, the genus Streptococcus, the genus Bifidobacterium, the genus Pediococcus, and the genus Saccaromyces. The bacterium of the genus Lactobacillus may be a bacterium of a species selected from the group consisting of Lactobacillus casei (L. casei), Lactobacillus acidophilus (L. acidophilus), Lactobacillus gasseri (L. gassen), Lactobacillus delbrueckii ssp bulgaricus (L. delbrueckii ssp bulgaricus), Lactobacillus helveticus (L. helveticus), Lactobacillus fermentum (L. fermentum), Lactobacillus paracasei (L. paracasei), Lactobacillus plantarum (L. plantarum), Lactobacillus reuteri (L. reuten), Lactobacillus rhamnosus (L. rhamnosus), and Lactobacillus salivarius (L. salivarius). For example, the probiotic may be Lactobacillus casei var. Rhamnosus. Lactobacillus casei var. Rhamnosus may also be classified as the species Lactobacillus Rhamnosus. The bacterium of the genus Lactococcus may be Lactococcus lactis (Lc. lactis). The bacterium of the genus Bacillus may be a bacterium of a species selected from the group consisting of Bacillus subtilis (B. subtilis), Bacillus coagulans (B. coagulans), Bacillus indicus (B. indicus), Bacillus clausii (B. clausii), Bacillus laterosporus, and Bacillus licheniformis (B. licheniformis). The bacterium of the genus Enterococcus may be Enterococcus faecium (E. faecium) or Enterococcus faecalis (E. faecalis). The bacterium of the genus Streptococcus may be Streptococcus thermophilus (S. thermophilus). The bacterium of the genus Bifidobacterium may be a bacterium of a species selected from the group consisting of Bifidobacterium bifidum (B. bifidum), Bifidobacterium breve (B. breve), Bifidobacterium dentium (B. dentium), Bifidobacterium longum (B. longum), Bifidobacterium infantis (B. infantis), and Bifidobacterium animalis (B. animalis). The bacterium of the genus Pediococcus is, for example, Pediococcus acidilactici (P. acidilactici). The bacterium of the genus Saccaromyces is, for example, Saccaromyces Boulardii.

The pharmaceutical composition may include at least two types of probiotics. The at least two types of probiotics may be for single or individual administration.

The term “antibiotics” refers to substances that inhibit the growth of microorganisms or kill microorganisms, and may be used interchangeably with the term “antimicrobial agent.” The antibiotics may include agents that act on bacteria, mold (fungi), and viruses. The antibiotic may be selected from the group consisting of a β-lactam antibiotic, a tetracycline antibiotic, an aminoglycoside antibiotic, a macrolid antibiotic, a glycopeptide antibiotic, a lincosamide antibiotic, and a quinolone antibiotic. The β-lactam antibiotic may be selected from a penicillin antibiotic, a cephalosporin antibiotic, and a monobactam antibiotic. The cephalosporin antibiotics may be divided into the 1^{st} to 4^{th} generations according to the antibacterial area and characteristics. The cephalosporin antibiotic may be selected from the group consisting of cefaclor (e.g., cefaclor hydrate), ceftazidime, cefoperazone, cefotaxime, ceftizoxime, ceftriaxone, cefepime, cefpirome, cefozopran, ceftaroline, ceftolozane, and ceftobiprole. The tetracycline antibiotic may be selected from the group consisting of doxycycline, tetracycline, chlortetracycline, oxytetracycline, demeclocycline, minocycline, lymecycline, methacycline, rolitetracycline, and tigecycline.

An injectable antibiotic such as ceftazidime may be finally used once alone after treatment of oral cefaclor and ramnos is repeated several times, to eliminate residual bacteria. While the combined treatment of cefaclor and ramnos is repeated, an injectable antibiotic may instead be injected alone into the ear. In addition, treatment may be repeated several times with a combination of ceftazidime and ramnos instead of cefaclor. In the case of rhinitis surgery to reduce inferior nasal concha, the ventilation function of an ear tube is improved, and thus, treatment progresses more quickly or there may be no recurrence after the treatment.

The term “inflammation” refers to a complex lesion that causes any one or more of tissue deterioration, circulatory disturbance and exudation, and tissue proliferation, as one of the protective responses of biological tissues to certain stimuli. The pharmaceutical composition may be a composition for preventing or treating inflammation caused by infection with a bacterium, a fungus, or a combination thereof. The bacterium includes gram-negative bacteria and gram-positive bacteria. The bacterium may be an antibiotic-resistant bacterium. The antibiotic-resistant bacterium may be selected from the group consisting of methicillin-resistant Staphylococcus aureus (MRSA), methicillin-sensitive Staphylococcus aureus (MSSA), vancomycin-resistant S. Aureus (VRSA), an extended-spectrum beta-lactamase (ESBL) bacterium, vancomycin-resistant Enterococcus (VRE), multidrug-resistant Acinetobacter baumannii (MRAB), multidrug-resistant Pseudomonas aeruginosa (MRPA), and carbapenem-resistant enterobacteriaceae.

The pharmaceutical composition may be for the prevention or treatment of a disease selected from the group consisting of otitis media, acute sinusitis, chronic sinusitis, pharyngitis, rhinitis, dermatitis, atopic dermatitis, bacterial infection or inflammation after surgery (e.g., plastic surgery), osteitis, osteomyelitis, otitis externa, necrotizing otitis externa, diabetic foot ulcer, intractable wound infection, inflammation in the abdominal cavity contaminated with feces, bacterial infection or inflammation at the site of insertion of foreign matter (e.g., silicone or Gore-tex), and intractable infectious diseases (e.g., tuberculosis infection, and inflammation in the cavity due to tuberculosis).

The term “prevention” refers to all actions that inhibit or delay the onset of inflammation via administration of the composition. The term “treatment” refers to all actions that alleviate or beneficially change the symptoms of inflammation via administration of the composition.

The pharmaceutical composition may be a composition for single administration or individual administration. The pharmaceutical composition may be a single or separate composition. The probiotic and the antibiotic may be separate compositions. The probiotic and the antibiotic may be a single composition of a mixture thereof. The pharmaceutical composition may be dry powder or a suspension.

The pharmaceutical composition may be in a topical dosage form. The topical dosage form may be an external preparation, drop, or a combination thereof. The external preparation may be an ointment, a cream, a lotion, a gel, a spray, a plaster, or the like. The drop may be an otic solution. In the case of otitis media, the pharmaceutical composition may be injected and added dropwise into the ear to be flowed into the ear. In the case of inflammation in a local site in the skin, the pharmaceutical composition may be applied or sprayed.

The pharmaceutical composition may include effective amounts of a probiotic and an antibiotic. The effective amounts may be appropriately selected depending on an individual. The effective amounts may be determined according to factors including the severity of disease, age, body weight, health, and gender of a patient, sensitivity of a patient to drugs, administration time, administration route, extraction rate, treatment periods, and drugs used in combination with or concurrently with the composition of the present invention, and other factors well known in the medical field. The effective amounts may be from about 0.01 g to about 1 g, from about 0.1 g to about 1 g, or from about 0.5 g to about 1 g, per 1 ml of the pharmaceutical composition.

The pharmaceutical composition may further include a carrier, an excipient, or a diluent. The carrier, the excipient, and the diluent may be, for example, physiological saline, lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or a combination thereof.

The dosage of the pharmaceutical composition may be, for example, from about 0.01 g to about 5 g, from about 0.1 g to about 4 g, from about 0.5 g to about 3 g, from about 0.5 g to about 2 g, or about 1 g, per adult. The pharmaceutical composition may be administered, for example, once a day, two to ten times a day, or every two days to every 1 week. The pharmaceutical composition may be administered, for example, twice, three times, four times, five times, six times, eight times, ten times, 12 times, or 15 or more times at intervals of two days, three days, four days, five days, 1 week, or 1 month.

Another embodiment provides a method of preventing or treating inflammation, including topically administering a probiotic and an antibiotic to an inflamed area of a subject.

The probiotic, the antibiotic, inflammation, prevention, and treatment are the same as described above.

The subject may be an individual with inflammation or having a risk of developing inflammation. The subject may be a mammal, for example, a human, a cow, a horse, a pig, a dog, sheep, a goat, or a cat.

The method may include simultaneously or sequentially administering a probiotic and an antibiotic. For example, a mixture of a probiotic and an antibiotic may be administered to the inflamed area of a patient. After a probiotic is administered to the inflamed area of a patient, an antibiotic may be administered.

The probiotic and the antibiotic may be administered, for example, in an amount of from about 0.01 g to about 5 g, from about 0.1 g to about 4 g, from about 0.5 g to about 3 g, from about 0.5 g to about 2 g, or about 1 g, per adult. The probiotic and the antibiotic may be administered, for example, once a day, two to ten times a day, or every two days to every 1 week. The probiotic and the antibiotic may be administered, for example, twice, three times, four times, five times, six times, eight times, ten times, 12 times, or 15 or more times at intervals of two days, three days, four days, five days, 1 week, or 1 month.

When administered to an inflamed area, the probiotic changes the environment of the inflamed area to inhibit the growth of bacteria and the like that cause inflammation or kill bacteria and the like, and may change the flora. The combinations of probiotics and antibiotics may prevent or treat inflammation by killing or inhibiting the growth of bacteria or fungi, for example, antibiotic-resistant bacteria.

### Advantageous Effects of Disclosure

A pharmaceutical composition for anti-inflammation, including a probiotic and an antibiotic, according to an embodiment, and a method using the same can efficiently prevent or treat an inflammatory disease.

### BRIEF DESCRIPTION OF DRAWINGS

FIG. 1A illustrates a result sheet for the examination of a microorganism in pus collected from otitis media patient 1, and FIG. 1B illustrates images of the inner ear of patient 1 before/after treatment.

FIGS. 2 to 7 are images of the inner ears before/after treatment of patient 2 to patient 7, respectively.

FIG. 8A illustrates inner ear images showing the eardrum of an otitis media patient before treatment, FIG. 8B illustrates images of the inner ears after treatment with cefaclor and lockstar capsules, FIG. 8C illustrates images of the inner ears after treatment with cefaclor and medilac-S capsules, and FIG. 8D illustrates final images of the inner ears after 1 week (F1: the inner ear without a lesion, F2: the inner ear with a lesion).

FIG. 9A is an inner ear image showing the eardrum of an otitis media patient before treatment, and FIG. 9B is an image of the inner ear of an otitis media patient after treatment with lockstar and doxycycline.

FIG. 10A illustrates images showing the nasal cavities of a sinusitis patient before treatment, and FIG. 10B illustrates images of the nasal cavities of a sinusitis patient after treatment with lockstar and cefaclor (F1: the nasal cavity without a lesion, F2: the nasal cavity with a lesion).

### MODE OF DISCLOSURE

Hereinafter, the present disclosure will be described in further detail with reference to the following examples. However, these examples are for illustrative purposes only and are not intended to limit the scope of the present disclosure.

### EXAMPLE 1

### Confirmation of Inflammation Treatment Effect of Composition Including Lactobacillus Strain and Antibiotic

To confirm whether a composition in which an antibiotic and a Lactobacillus strain as a probiotic are mixed is effective in treating inflammation, the efficacy was evaluated clinically.

First, the contents of 1 capsule of ramnos for oral use (Han Wha Pharma Co., Ltd., ingredient name: 250 mg of a freeze-dried culture of Lactobacillus casei var. Rhamnosus) and 1 capsule of fronclor (Han Wha Pharma Co., Ltd., ingredient name: 262 mg of cefaclor hydrate) were mixed in a number ratio of 1:1, and physiological saline was added to prepare about 1.5 ml of a mixture.

Case 1) Patient 1 (male/59 years old)

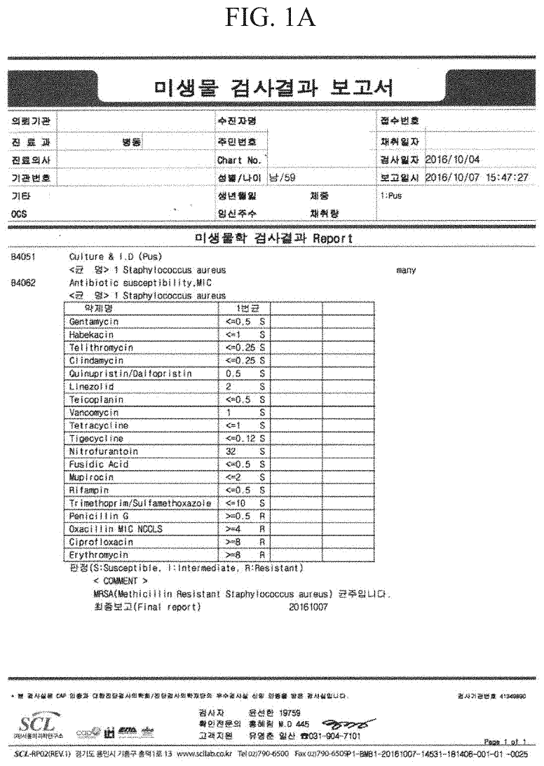

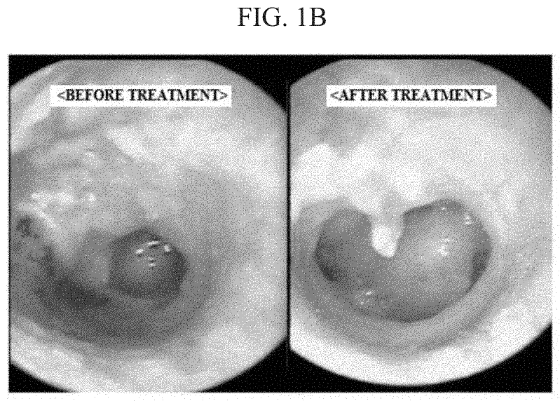

The patient had been suffering from otitis media for more than several years because there was no treatment effect even when the patient took antibiotics including cefaclor at other hospitals. Pus was collected from the ear of the patient, and a microorganism in the pus was examined. The microorganism examination results are illustrated in FIG. 1A.

As illustrated in FIG. 1A, the patient was infected with Staphylococcus aureus, and the bacterium was methicillin-resistant Staphylococcus aureus (MRSA), which has resistance to an antibiotic. Thus, it was confirmed that the patient was infected with a multidrug-resistant bacterium.

The prepared mixture was injected into an ear of the patient via a syringe or added dropwise thereinto to be flowed into the ear. After this treatment was repeated three times at intervals of one to three days, a lesion was confirmed. Images before/after treatment are illustrated in FIG. 1 B. As illustrated in FIG. 1 B, fungi and ooze on the left side disappeared, from which it was confirmed that inflammation was treated. In addition, there was no recurrence even after several months after treatment, from which it was determined that the inflammation was completely cured.

Case 2) Patient 2 (female/90 years old)

The patient was diagnosed with otitis media and had fungi and ooze in the inner ear.

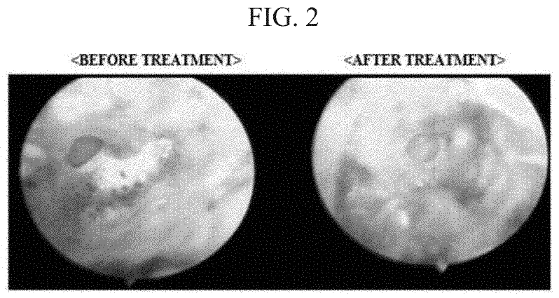

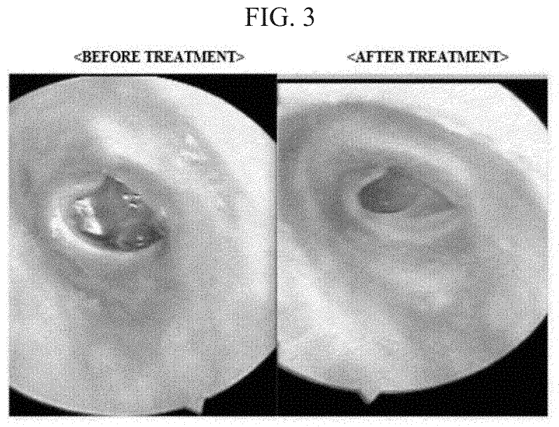

As described above, the prepared mixture was injected with a syringe or added dropwise to be flowed into the ear. After this treatment was repeated about five times, a lesion was confirmed. Images before/after treatment are illustrated in FIG. 2. As illustrated in FIG. 2, it was confirmed that fungi and ooze on the left side disappeared.

Case 3) Patient 3 (male/78 years old)

The patient was diagnosed with otitis media and had ooze and symptoms such as bubbles in the inner ear. The patient was infected with Staphylococcus aureus, and the bacterium was methicillin-sensitive Staphylococcus aureus (MSSA), which has resistance to an antibiotic.

As described above, the prepared mixture was injected with a syringe or added dropwise to be flowed into the ear. After this treatment was repeated three times, a lesion was confirmed. Images before/after treatment are illustrated in FIG. 3. As illustrated in FIG. 3, it was confirmed that ooze and symptoms such as bubbles on the left side disappeared.

Case 4) Patient 4 (female/59 years old)

The patient was diagnosed with otitis media, and had a lump of ooze in the inner ear.

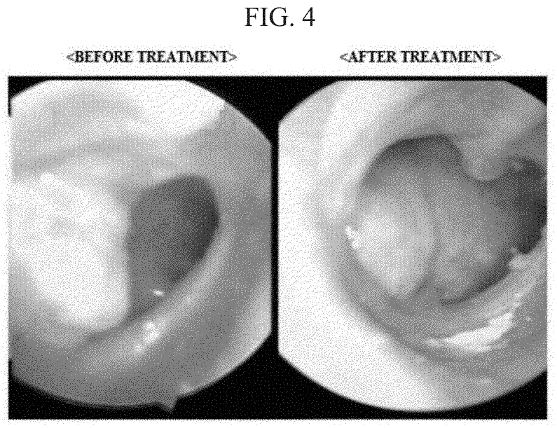

As described above, the prepared mixture was injected with a syringe or added dropwise to be flowed into the ear. After this treatment was repeated 12 times at intervals of one to three days, a lesion was confirmed. Images before/after treatment are illustrated in FIG. 4. As illustrated in FIG. 4, it was confirmed that the lump of ooze on the left side disappeared.

Case 5) Patient 5 (male/77 years old)

The patient was diagnosed with otitis media, and had fungi and ooze in the inner ear. Through bacterial examination, Staphylococcus lugdunensis was confirmed, and this bacterium showed susceptibility to all antibiotics except for penicillin.

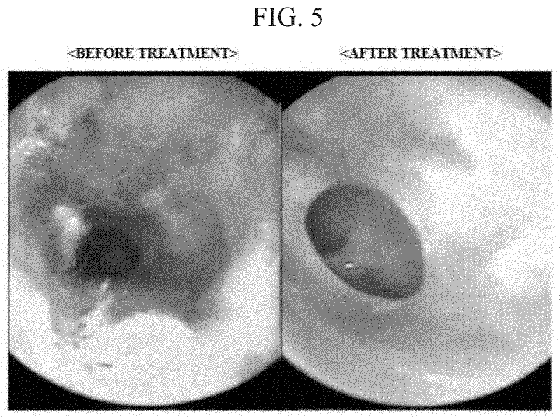

As described above, the prepared mixture was injected with a syringe or added dropwise to be flowed into the ear. After this treatment was repeated four times at intervals of one to three days, a lesion was confirmed. Images before/after treatment are illustrated in FIG. 5. As illustrated in FIG. 5, it was confirmed that fungi and ooze on the left side disappeared.

Case 6) Patient 6 (female/75 years old)

The patient was diagnosed with severe otitis media and necrotizing otitis externa, and had a lot of ooze in the inner ear.

About 1 ml of an extract, obtained from 1 g of 1 vial of ceftazidime injection (ceftazidime injection, UNION KOREA PHARM CO., LTD.) mixed with 3 ml of physiological saline, was mixed again with 1 capsule of ramnos (Han Wha Pharma Co., Ltd., ingredient name: 250 mg of a freeze-dried culture of Lactobacillus casei var. Rhamnosus) to prepare about 1.5 ml of a mixture.

The mixture was injected with a syringe or added dropwise to be flowed into the ear. After this treatment was repeated seven times at intervals of one to three days, ooze disappeared, but recurred after two days. Thus, the contents of 1 capsule of ramnos for oral use (Han Wha Pharma Co., Ltd., ingredient name: 250 mg of a freeze-dried culture of Lactobacillus casei var. Rhamnosus) and 1 capsule of fronclor (Han Wha Pharma Co., Ltd., ingredient name: 262 mg of cefaclor hydrate) were mixed in a number ratio of 1:1, and physiological saline was added to prepare about 1.5 ml of a mixture, and the mixture was injected into the middle ear cavity via the external auditory canal four times at intervals of one to three days. No oozing occurs any more. However, to eliminate any remaining bacteria, after two days, 1 ml of 1 vial of ceftazidime injection (1 g, ceftazidime injection, UNION KOREA PHARM CO., LTD.) mixed with 3 ml of physiological saline was injected alone into the ear.

After three days, clear ooze was observed, but not pus. Thus, the contents of 1 capsule of ramnos for oral use (Han Wha Pharma Co., Ltd., ingredient name: 250 mg of a freeze-dried culture of Lactobacillus casei var. Rhamnosus) and 1 capsule of fronclor (Han Wha Pharma Co., Ltd., ingredient name: 262 mg of cefaclor hydrate) were mixed in a number ratio of 1:1, and physiological saline was added to prepare about 1.5 ml of a mixture. The mixture was injected into the middle ear cavity via the external auditory canal three times at intervals of three days, and a lesion was confirmed.

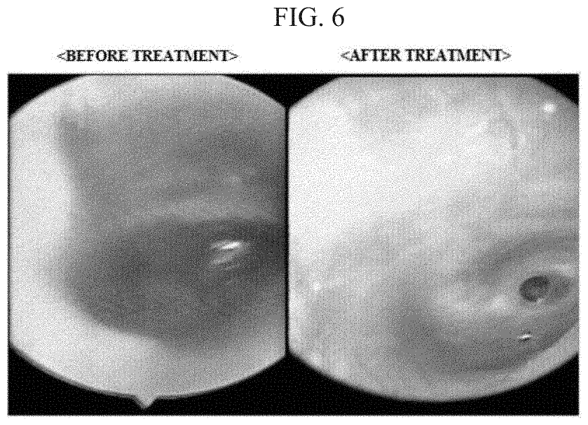

Images before/after treatment are illustrated in FIG. 6. As illustrated in FIG. 6, a lot of stagnant ooze on the left side completely disappeared and a perforation in the eardrum was seen.

Case 7) Patient 7 (female/88 years old)

The patient was diagnosed with otitis media and had symptoms such as bubbles in the inner ear. The patient was infected with Staphylococcus aureus, and the bacterium was methicillin-sensitive Staphylococcus aureus (MSSA).

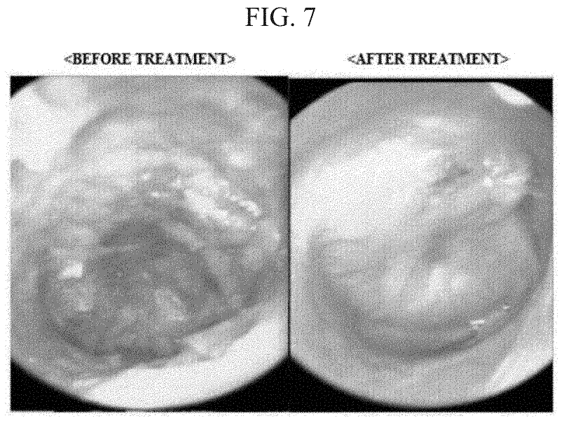

As described above, the prepared mixture was injected with a syringe or added dropwise to be flowed into the ear. After this treatment was repeated three times at intervals of one day, a lesion was confirmed. Images before/after treatment are illustrated in FIG. 7. As illustrated in FIG. 7, it was confirmed that ooze and symptoms such as bubbles on the left side disappeared.

### EXAMPLE 2

### Confirmation of Inflammation Treatment Effect of Composition Including Probiotic and Antibiotic

2-1. Inflammation Treatment Effect of Probiotic and Cefaclor

To confirm whether other probiotics in addition to Lactobacillus casei var. Rhamnosus and cefaclor also have an inflammation treatment effect, a rockstar capsule for oral use (KOLON PHARMA, ingredient name: 100 mg of a freeze-dried culture of Lactobacillus casei var. Rhamnosus) and a medilac-S enteric capsule for oral use (Hanmi, Ingredient name: Bacillus subtilis and Enterococcis faecium) were prepared.

Physiological saline was added to a mixture of 1 capsule of rockstar and 1 capsule of fronclor to prepare about 3 ml of a mixture. As described in Example 1, the prepared mixture was injected into the middle ear of a patient with otitis media. As a result of follow-up of the patient with otitis media, it was confirmed that inflammation due to otitis media was ameliorated and the eardrum was also narrowed. Subsequently, the same process was repeated using the medilac-S capsule instead of the rockstar capsule.

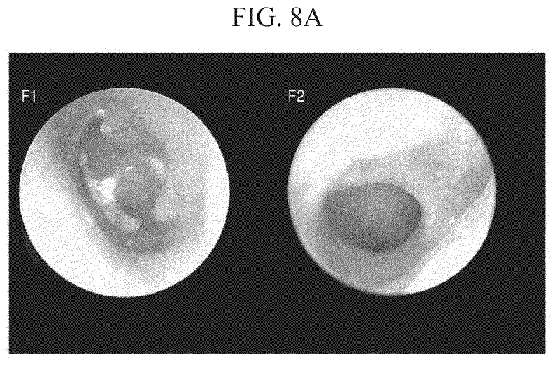

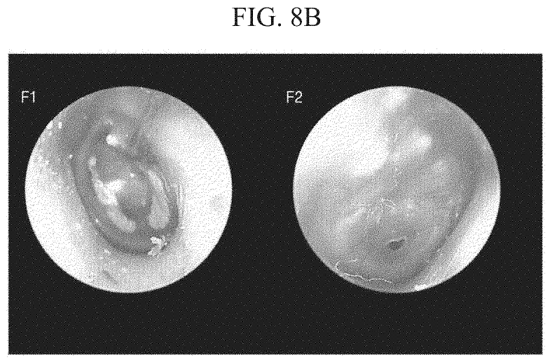

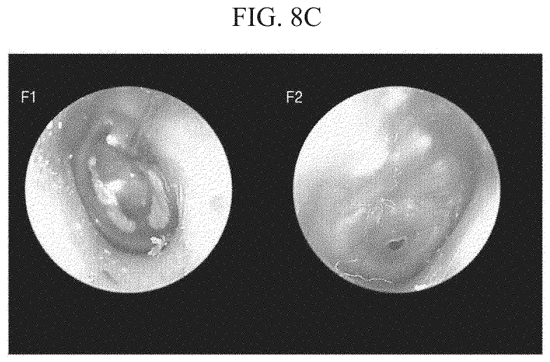

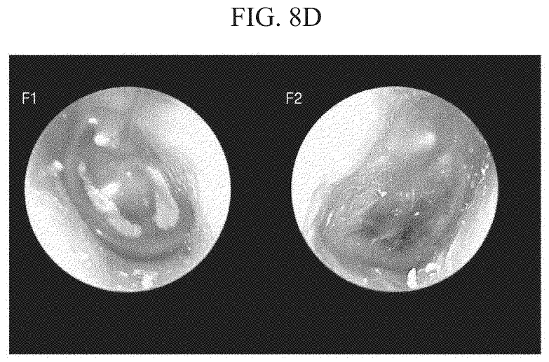

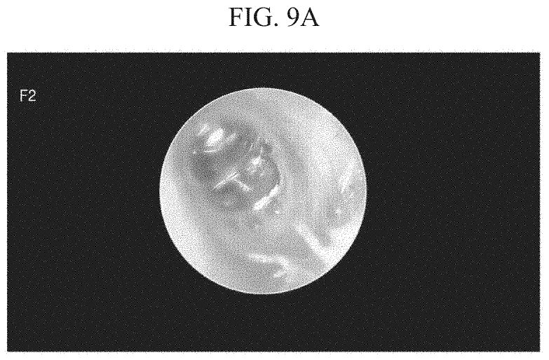

Images showing the eardrum of a patient before treatment are illustrated in FIG. 8A, images after treatment with cefaclor and the rockstar capsule are illustrated in FIG. 8B, and images after the subsequent treatment with cefaclor and the medilac-S capsule are illustrated in FIG. 8C, and the final images after one week are illustrated in FIG. 8D.

As illustrated in FIGS. 8A to 8D, it was confirmed that, through the combination of a probiotic and an antibiotic, a perforation in the eardrum was narrowed or completely closed, and inflammation disappeared.

2-2. Inflammation Treatment Effect of Probiotic and Doxycycline

To confirm whether other combinations besides a probiotic and cefaclor also have an inflammation treatment effect, a rockstar capsule and doxycycline (doxyramycin capsules 100 mg, manufacturer: SINIL Pharmaceutical Co., Ltd, seller: DongKoo Bio&Pharma Co., Ltd.) were prepared.

Physiological saline was added to a mixture of 1 capsule of rockstar and 1 capsule of doxycycline to prepare about 3 ml of a mixture. As described in Example 1, the prepared mixture was injected into the middle ear of a patient with otitis media.

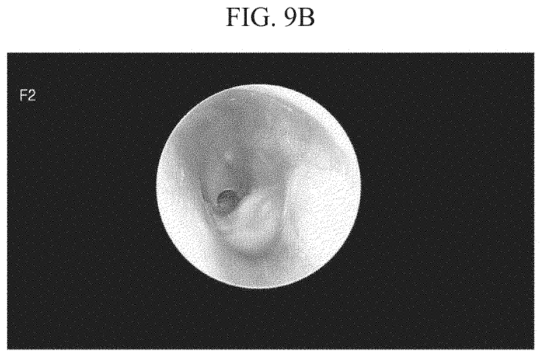

An image showing the eardrum of the patient before treatment is illustrated in FIG. 9A, and an image after treatment with rockstar and doxycycline is illustrated in FIG. 9B.

As illustrated in FIGS. 9A and 9B, it was confirmed that, even when doxycycline, which is a different class from cefaclor, was used as an antibiotic, a perforation in the eardrum was narrowed or completely closed, and inflammation was treated.

### EXAMPLE 3

### Confirmation of Effect of Composition Including Probiotic and Antibiotic on Treating Sinusitis

To confirm whether a composition in which a probiotic and an antibiotic were mixed also has a therapeutic effect on other inflammations as well as otitis media, patients who also has sinusitis were selected from among patients with otitis media.

For the selected patients, the inside of a nose was washed in the same manner as in Example 1.

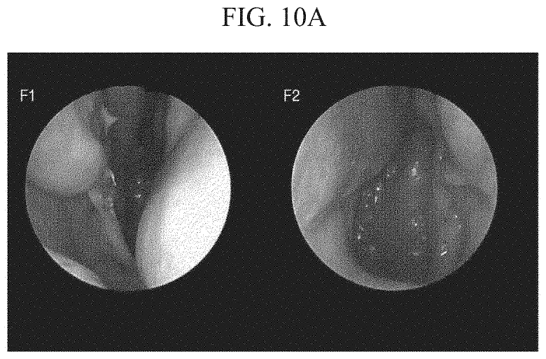

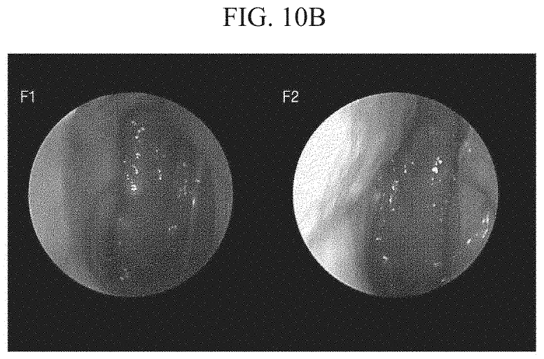

Images showing the nasal cavities of the patients before treatment are illustrated in FIG. 10A, and images after treatment with rockstar and cefaclor are illustrated in FIG. 10B.

As illustrated in FIGS. 10A and 10B, it was confirmed that symptoms of sinusitis completely disappeared by using a probiotic and an antibiotic.

As shown in Examples 1 to 3, it has been confirmed that a composition including a probiotic and an antibiotic is effective in the prevention or treatment of inflammation. Therefore, it has been confirmed that, in inflammation caused by bacterial or fungal infection, the inflammation can be prevented or treated by replacing the flora of bacteria or fungi with bacteria of probiotics and killing bacteria by using antibiotics.

1. A method of preventing or treating inflammation, the method comprising locally administrating a probiotic and an antibiotic to an inflamed area of a subject.

2. The method of claim 1, wherein the probiotic is a bacterium of a genus selected from the group consisting of the genus Lactobacillus, the genus Lactococcus, the genus Bacillus, the genus Enterococcus, the genus Streptococcus, the genus Bifidobacterium, the genus Pediococcus, and the genus Saccaromyces.

3. The method of claim 2, wherein the bacterium of the genus Lactobacillus is a bacterium of a species selected from the group consisting of Lactobacillus casei (L. casei), Lactobacillus acidophilus (L. acidophilus), Lactobacillus gasseri (L. gasseri), Lactobacillus delbrueckii ssp bulgaricus (L. delbrueckii ssp bulgaricus), Lactobacillus helveticus (L. helveticus), Lactobacillus fermentum (L. fermentum), Lactobacillus paracasei (L. paracasei), Lactobacillus plantarum (L. plantarum), Lactobacillus reuteri (L. reuteri), Lactobacillus rhamnosus (L. rhamnosus), and Lactobacillus salivarius (L. salivarius).

4. The method of claim 2, wherein the bacterium of the genus Bacillus is a bacterium of a species selected from the group consisting of Bacillus subtilis (B. subtilis), Bacillus coagulans (B. coagulans), Bacillus indicus (B. indicus), Bacillus clausii (B. clausii), Bacillus laterosporus, and Bacillus licheniformis (B. licheniformis).

5. The method of claim 2, wherein the bacterium of the genus Enterococcus is a bacterium of a species selected from the group consisting of Enterococcus faeciurn (E. faecium) and Enterococcus faecalis (E. faecalis).

6. The method of claim 1, wherein the antibiotic is selected from the group consisting of a β-lactam antibiotic, a tetracycline antibiotic, an aminoglycoside antibiotic, a macrolid antibiotic, a glycopeptide antibiotic, a lincosamide antibiotic, and a quinolone antibiotic.

7. The method of claim 6, wherein the β-lactam antibiotic is selected from the group consisting of a penicillin antibiotic, a cephalosporin antibiotic, and a monobactam antibiotic.

8. The method of claim 7, wherein the cephalosporin antibiotic is selected from the group consisting of cefaclor, ceftazidime, cefoperazone, cefotaxime, ceftizoxime, ceftriaxone, cefepime, cefpirome, cefozopran, ceftaroline, ceftolozane, and ceftobiprole.

9. The method of claim 6, wherein the tetracycline antibiotic is selected from the group consisting of doxycycline, tetracycline, chlortetracycline, oxytetracycline, demeclocycline, minocycline, lymecycline, methacycline, rolitetracycline, and tigecycline.

10. The method of claim 1, wherein the pharmaceutical composition is for preventing or treating inflammation is caused by infection with a bacterium, a fungus, or a combination thereof.

11. The method of claim 10, wherein the bacterium is an antibiotic-resistant bacterium selected from the group consisting of methicillin-resistant Staphylococcus aureus (MRSA), methicillin-sensitive Staphylococcus aureus (MSSA), vancomycin-resistant S. aureus (VRSA), an extended-spectrum beta-lactamase (ESBL) bacterium, vancomycin-resistant Enterococcus (VRE), multidrug-resistant Acinetobacter baumannii (MRAB), multidrug-resistant Pseudomonas aeruginosa (MRPA), and carbapenem-resistant enterobacteriaceae.

12. The method of claim 1, wherein the inflammation is associated with a disease selected from the group consisting of otitis media, acute sinusitis, chronic sinusitis, pharyngitis, rhinitis, dermatitis, atopic dermatitis, bacterial infection or inflammation after surgery, osteitis, osteomyelitis, otitis externa, necrotizing otitis externa, diabetic foot ulcer, intractable wound infection, inflammation in an abdominal cavity contaminated with feces, bacterial infection or inflammation at a site of insertion of foreign matter, and intractable infectious diseases.

13. The method of claim 1, wherein the method is for single administration or individual administration.

14. The method of claim 1, wherein the method is an external preparation, drop, or a combination thereof.

15. (canceled)

16. The method of claim 1, wherein the probiotic and the antibiotic are simultaneously or sequentially administered.
